(19) United States
(12) Patent Application Publication
(10) Pub. No.: US 2014/0257076 A1
(43) Pub. Date: Sep. 11, 2014

(54) NON-CONTACT OPHTHALMOLOGIC APPARATUS, CONTROL METHOD FOR THE SAME, PROCESSING APPARATUS, PROCESSING METHOD, AND NON-TRANSITORY TANGIBLE MEDIUM HAVING A PROGRAM RECORDED THEREON

(71) Applicant: CANON KABUSHIKI KAISHA, Tokyo (JP)

(72) Inventor: Yuki Shimozato, Kawasaki-shi (JP)

(73) Assignee: CANON KABUSHIKI KAISHA, Tokyo (JP)

(21) Appl. No.: 14/190,487

(22) Filed: Feb. 26, 2014

(30) Foreign Application Priority Data
Mar. 11, 2013 (JP) 2013-047759

(51) Int. Cl. A61B 3/16

(52) U.S. Cl. CPC A61B 3/165
USPC 600/401

(57) ABSTRACT

A non-contact ophthalmologic apparatus includes: a storage unit for storing measurement results of an intraocular pressure of an eye to be inspected as statistical data; a condition determination unit for determining a measurement range of an intraocular pressure value based on the statistical data; and a control condition determination unit for determining, based on the measurement range determined by the condition determination unit, a control condition of a fluid ejection unit for ejecting air toward the eye to be inspected. Thus, it is possible to eliminate a need to eject high-pressure air even in the first measurement of the intraocular pressure of the eye to be inspected, thereby alleviating a burden on a subject.

### BACKGROUND OF THE INVENTION

1. Field of the Invention

The present invention relates to a non-contact ophthalmologic apparatus for calculating an intraocular pressure value based on a corneal deformation detection signal obtained by ejecting air toward an eye to be inspected, and to a control method for the non-contact ophthalmologic apparatus. Further, the present invention relates to a processing apparatus and a processing method for calculating an intraocular pressure value, and to a non-transitory tangible medium having a program, recorded thereon.

2. Description of the Related Art

Most non-contact tonometers of ophthalmologic apparatus are configured so that a piston pushes air inside a cylinder to compress the air inside an air chamber, and the compressed air is ejected through a nozzle. The piston is generally driven through use of a rotary solenoid because of its strong initial torque and long stroke. When the cornea is then deformed into a predetermined state, control is executed to turn OFF the power supply to the solenoid. However, even after the power supply to the solenoid is turned OFF, the piston continues to move for a while due to an inertial force. Therefore, excessive air is ejected toward the subject, resulting in an excessive burden on the subject. To minimize the excessive air, it is necessary to reduce the inertial force of the piston.

In general, normal eyes have an intraocular pressure value of 10 mmHg to 20 mmHg, but eyes suffering from a disease such as glaucoma have a high intraocular pressure of 20 mmHg or more. Therefore, the tonometer needs to have a wide measurement range of about 0 mmHg to 60 mmHg. As the upper limit of the measurement range is higher, the maximum pressure of the ejected air needs to be higher as well, and hence the piston needs to be moved at higher speed. Thus, as the upper limit is higher, the inertial force of the piston becomes more significant, resulting in ejection of excessive air. As a method of solving this problem, Japanese Patent Application Laid-open No. S63-300740 proposes such a method that the maximum pressure of a compressed fluid may be set at multiple levels, and the maximum pressure necessary for the subsequent measurement is selected therefrom based on information of the measured intraocular pressure value. Further, Japanese Patent No. 3,168,014 proposes such a method that the maximum pressure of ejected air in the subsequent measurement is set based on a state of the corneal deformation detection signal obtained in the previous measurement.

### SUMMARY OF THE INVENTION

According to one embodiment of the present invention, there is provided a non-contact ophthalmologic apparatus, including: a fluid ejection unit for ejecting compressed air toward a cornea of an eye to be inspected; an intraocular pressure measurement unit for measuring an intraocular pressure by detecting a state in which the cornea is deformed; a storage unit for storing measurement results of the intraocular pressure as statistical data; a first determination unit for determining a measurement range of the intraocular pressure based on the statistical data; and a second determination unit for determining a control condition of the fluid ejection unit based on the determined measurement range.

Further features of the present invention will become apparent from the following description of exemplary embodiments with reference to the attached drawings.

### BRIEF DESCRIPTION OF THE DRAWINGS

FIG. 1 is a configuration diagram of a non-contact tonometer according to an embodiment of the present invention.

FIG. 2 is a graph showing a relationship between a current value of a solenoid and a pressure of ejected air,

FIG. 3 is a system configuration diagram of the non-contact tonometer according to the embodiment of the present invention.

FIGS. 4A and 4B are explanatory histograms showing a determination method for an upper limit value of a measurement range.

FIG. 5 is a flow chart of determination of the measurement range to be executed through a single measurement.

FIG. 6 is a diagram illustrating a measurement screen showing the measurement range.

FIG. 7 is a flow chart of determination of the measurement range to be executed through N measurements.

### DESCRIPTION OF THE EMBODIMENTS

The intraocular pressure value of an eye to be inspected is not confirmed before measurement, and hence, in the related art, it is necessary to eject air at the maximum pressure within a measurement range in the first measurement so as to prevent a measurement error that may be caused by an insufficient pressure of the ejected air. Therefore, in the first measurement, a significant burden may be imposed on the subject. Further, depending on hospitals where the apparatus is installed, for example, in a case of hospitals where mass-screening tests such as a diagnostic test are conducted frequently, it may not be preferred to repeat measurement that is executed multiple times for each subject.

It is therefore an object of an embodiment of the present invention to reduce ejection of excessive air even in the first measurement, thereby alleviating a burden on the subject.

A non-contact ophthalmologic apparatus or a processing apparatus according to this embodiment is configured to store measurement results of an intraocular pressure as statistical data, determine a measurement range of the intraocular pressure based on the statistical data, and to determine a control condition of a fluid ejection unit based on the determined measurement range.

Thus, the measurement range may be determined based on the statistical data on the measurement results. Therefore, even in the first measurement, the air may be ejected at a pressure suitable for characteristics of the eye of the subject to be inspected, specifically, the intraocular pressure of the eye to be inspected. As a result, the burdens on many subjects may be alleviated.

Now, the embodiment of the present invention is described in detail with reference to the drawings.

(Embodiment)

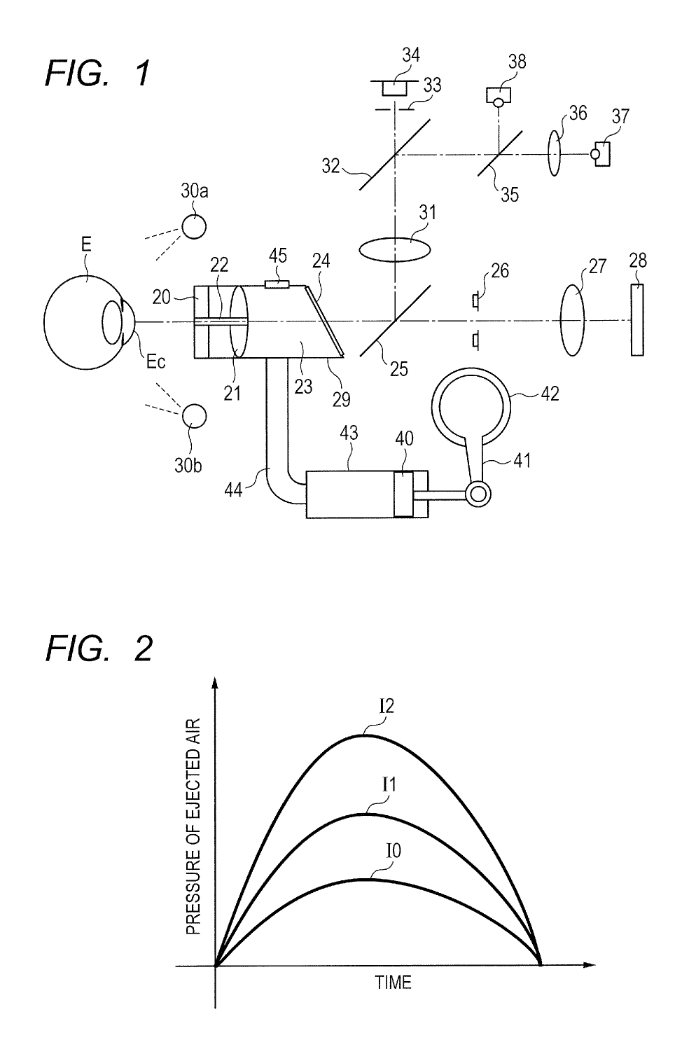

FIG. 1 is a schematic configuration diagram illustrating a non-contact tonometer according to an embodiment of the present invention.

First, an optical system of this tonometer is described. A measurement light source 37 is a near-infrared LED to be used for both measurement and alignment with an. eye E to be inspected. In a light emitting direction of the measurement light source 37, a projection lens 36, a half-silvered mirror 35, and a half-silvered mirror 32 are arranged. In a light reflecting direction of the half-silvered mirror 35 as seen from the half-silvered mirror 32, a fixation light source 38 is arranged as an LED to be fixated by a subject. In a light reflecting direction of the half-silvered mirror 32 as seen from the half-silvered mirror 35, a relay lens 31 is arranged.

A nozzle 22 is arranged on a center axis of a plane parallel glass 20 and an objective lens 21 so as to be opposed to a cornea Ec of the eye E to be inspected. Behind the nozzle 22, an air chamber 23, an observation window 24; a dichroic mirror 25, a prism stop 26, an imaging lens 27, and an image pickup element 28 are arranged sequentially. Those components constitute a light receiving optical path and an alignment detecting optical path of an observation optical system for the eye E to be inspected.

The plane parallel glass 20 and the objective lens 21 are supported by an objective lens barrel 29, and anterior ocular segment illumination light sources 30a and 30b for illuminating the eye E to be inspected are arranged outside the objective lens barrel 29. Note that, the anterior ocular segment illumination light sources 30a and 30b are illustrated on the upper and lower sides of FIG. 1 for convenience of description, but in actuality, the anterior ocular segment illumination light sources 30a and 30b are arranged to be opposed to an optical axis in a direction perpendicular to the drawing sheet.

In a light entering direction of the half-silvered mirror 32 as seen from the relay lens 31, an aperture 33 and a light receiving element 34 are arranged. Note that, the aperture 33 is arranged at a position conjugate to a cornea reflection image of the measurement light source 37 when the cornea Ec is deformed into a predetermined shape, and constitutes, together with the light receiving element 34, a light receiving optical system for detecting deformation of the cornea Ec in a direction of a visual axis. The relay lens 31 is designed so that a cornea reflection image having substantially the same size as the aperture 33 is formed at the position of the aperture 33 when the cornea Ec is deformed into the predetermined shape. Those components constitute an intraocular pressure measurement unit of this embodiment together with a system control portion including a calculation processing portion and the like to be described later.

Next, a mechanism for ejecting air in this apparatus is described. A pressure sensor 45 for monitoring an internal pressure of the air chamber 23 and a transfer tube 44 for feeding compressed air from a cylinder 43 are connected to the inside of the air chamber 23. A piston 40 is fitted to the cylinder 43, and is driven by a solenoid 42. Rotary motion of the solenoid 42 is converted into linear motion of the piston 40 by a drive lever 41 connected between the solenoid 42 and the piston 40. When the piston 40 moves inside the cylinder 43 at high speed, the air inside the cylinder 43 is fed to the air chamber 23 through the transfer tube 44, and thus the compressed air is ejected to the eye E to be inspected through the nozzle 22. Note that, the above-mentioned configuration for ejecting the compressed air toward the cornea of the eye to be inspected, corresponds to a fluid ejection unit of this embodiment.

Now, a control method for the solenoid at the time when a measurement range is changed is described. FIG. 2 shows a relationship between a value of a current allowed to flow through the solenoid and a pressure of ejected air. When the value of the current allowed to flow through the solenoid is increased at three levels, the air is ejected at pressures 10, 11, and 12, respectively. In this manner, the maximum pressure of the ejected air is increased. Thus, through the change of the current value, the measurement range is changed. Note that, the current value of the solenoid is herein taken as an example of the control condition of the fluid ejection unit of this embodiment, but a voltage, a current waveform, or the like may also be used as the control condition as long as the pressure of the ejected air may be controlled. Further, in this embodiment, the solenoid is used as a drive device of the piston 40, but a device other than the solenoid may be used for driving the piston 40. In this case, parameters for controlling this device are used as the control condition.

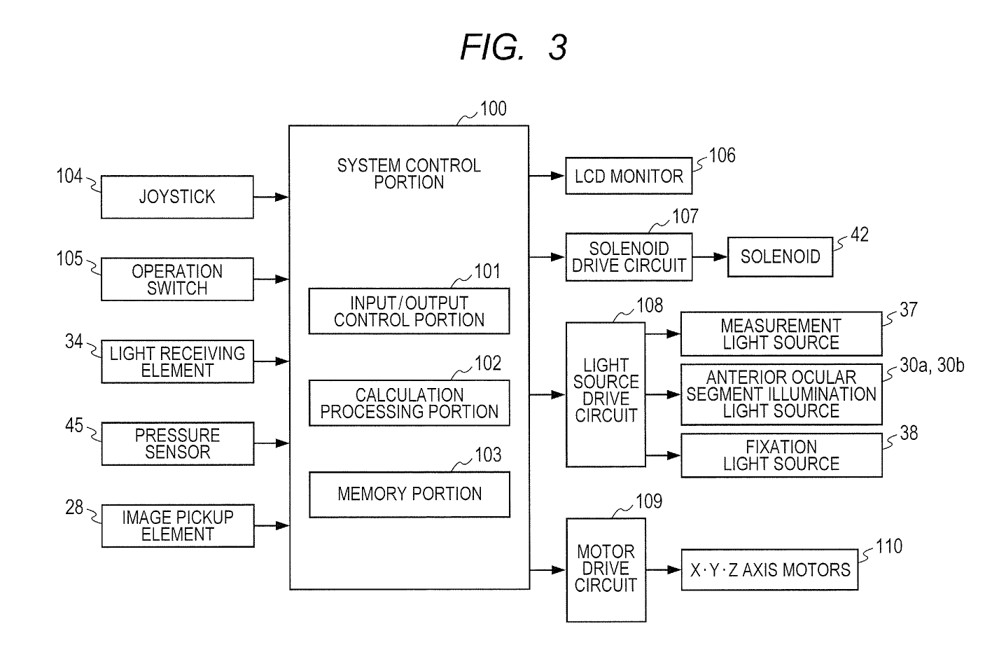

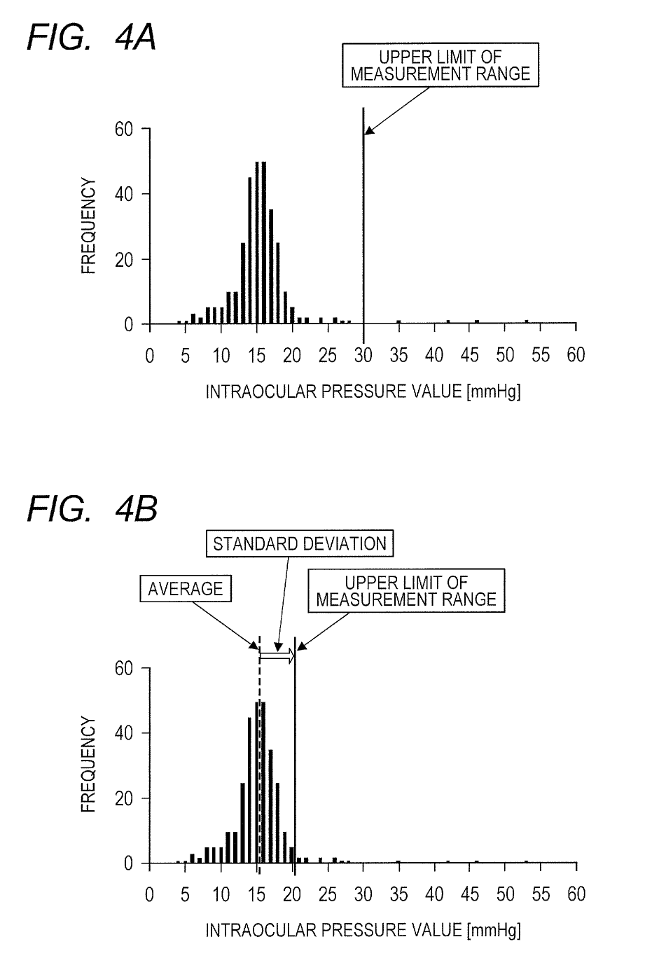

Next, a system configuration of this apparatus illustrated in FIG. 3 is described. A system control portion 100 for controlling the entire system includes an input/output control portion 101 for controlling input/output between the system control portion 100 and various devices, a calculation processing portion 102 for calculating data obtained from the various devices, and a memory portion 103 for storing a program, a measurement range, and data obtained through measurement. The system control portion 100 is connected to a joystick 104 to be used for aligning the position with the eye E to be inspected, an operation switch 105 to be used for starting measurement, the light receiving element 34 for receiving light of the cornea reflection image, the pressure sensor 45 for measuring the pressure inside the air chamber, and to the image pickup element 28 to be used for observing an image of an anterior ocular segment. Thus, signals are input from those components to the system control portion 100. Further, the system control portion 100 is connected to an LCD monitor 106 for displaying a screen, a solenoid drive circuit 107 for driving the piston, a light source drive circuit 108 for controlling ON/OFF of the light source, and to a motor drive circuit 109 for driving the measurement portion. Thus, signals are output from the system control portion 100 to those components.

Next, determination of the measurement range of this embodiment is described in detail with reference to FIGS. 4A and 4B.

FIGS. 4A and 4B are histograms showing an intraocular pressure value of 15 mmHg on average, which is measured through use of the apparatus installed at a given place. The horizontal axis represents an intraocular pressure value, and the vertical axis represents a frequency. FIG. 4A shows an upper limit line in a case where the measurement range is determined as a measurement range for low intraocular pressure that allows measurement of up to 30 mmHg. The ratio of eyes having an intraocular pressure value of 21 mmHg to 30 mmHg is lower than that of eyes having an intraocular pressure value of 0 mmHg to 20 mmHg. In the measurement range for low intraocular pressure, excessive air is ejected toward the eyes of the people having the intraocular pressure value of 0 mmHg to 20 mmHg who are majority of population so as to allow measurement also for the people having the intraocular pressure value of 21 mmHg to 30 mmHg who are minority of population. In this case, the upper limit of the measurement range is decreased, and thus a smaller amount of air may be ejected toward many people as compared to the related art, with the result that the burden on the people may be alleviated.

Characteristics of distribution of the measured intraocular pressure values differ depending on hospitals. For example, contact lens clinics frequently deal with eyes having an intraocular pressure value of 20 mmHg or less. In hospitals where the first medical examinations are frequently conducted for patients suffering from glaucoma with nigh intraocular pressure, on the other hand, the hospitals may frequently deal with eyes having a high intraocular pressure value. Thus, the optimum upper limit of the measurement range differs among hospitals. This embodiment has a feature in that the upper limit of the measurement range is determined so as to be suitable for the characteristics of the hospital where the apparatus is installed. Note that, in this determination of the measurement range, the following configuration is also preferred. For example, values corresponding to the types of hospital or the like are stored in advance, and a selection unit for selecting the type of hospital at the time of actual measurement is arranged. Through the selection of the type of hospital by the selection unit, the measurement range corresponding to this type of hospital is selected and determined.

In this case, it is preferred that a selection unit for selecting one of multiple types of hospital be further arranged in this apparatus. Further, along with this, the memory portion 103 stores multiple pieces of statistical data corresponding to the multiple types of hospital. Thus, a first determination unit described later is configured to determine the statistical data corresponding to the selected type, and to determine the measurement range of the intraocular pressure of the eye to be inspected based on the determined statistical data.

A histogram of the previously measured intraocular pressure values is stored in the apparatus. Then, statistical data of an average and a standard deviation is calculated based on the histogram. An optimum measurement range is determined based on the statistical data, FIG. 4B shows an upper limit line in a case where the upper limit value of the measurement range is determined as a value obtained by adding the standard deviation to the average. It is understood that the measurement range has a low upper limit and allows measurement for many people. That is, the histogram is obtained based on multiple measurement values, and the statistical data includes the average and the standard deviation that are obtained based on the histogram. The control condition of the fluid ejection is determined, as such a condition that the intraocular pressure value does not exceed the upper limit value of the measurement range obtained by adding the standard deviation to the average.

A procedure of measurement of this embodiment is described. In the measurement, positional alignment is first executed. An examiner depresses the operation switch 105 under a state in which the fixation light source 38 is fixated by the eye E to be inspected. When the operation switch 105 is depressed, the measurement light source 37 for the intraocular pressure is turned ON. The light beam emitted from the measurement light source 37 for the intraocular pressure is collimated by the projection lens 36, and is reflected by the half-slivered mirror 32. Then, the light beam is temporarily imaged inside the nozzle 22 by the relay lens 31, and is radiated onto the cornea Ec of the eye E to be inspected. A cornea bright spot formed by the cornea Ec is divided by the prism stop 26, and is imaged on the image pickup element 28. The system control portion 100 combines an image output from the image pickup element 28 with character and graphical data, and the resultant image is displayed on the LCD monitor 106. Based on a positional relationship between the split corneal bright spots, the system control portion 100 drives a main body drive motor 110 to automatically execute positional alignment in directions of the X-, Y-, and Z-axes. Note that, the positional alignment may be executed manually. In this case, the examiner operates the joystick 104 while observing the corneal bright spots displayed on the LCD monitor 106, and the system control portion 100 drives the main body drive motor 110 based on the input through the joystick 104. The positional alignment is completed, when the positional relationship between the corneal bright spots is brought into a predefined state. When the positional alignment is completed, measurement of the intraocular pressure is started.

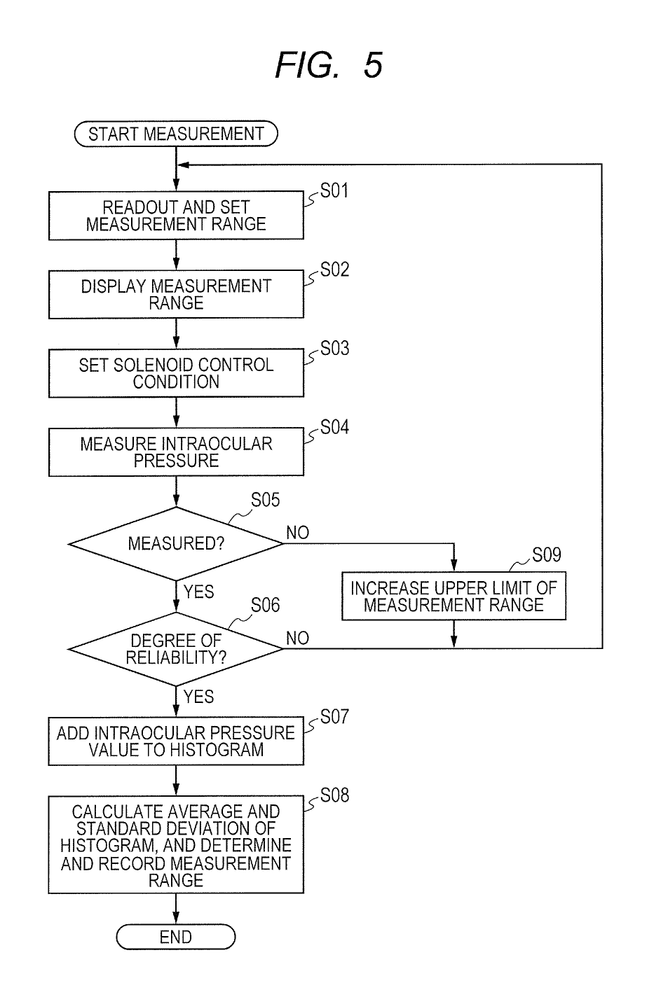

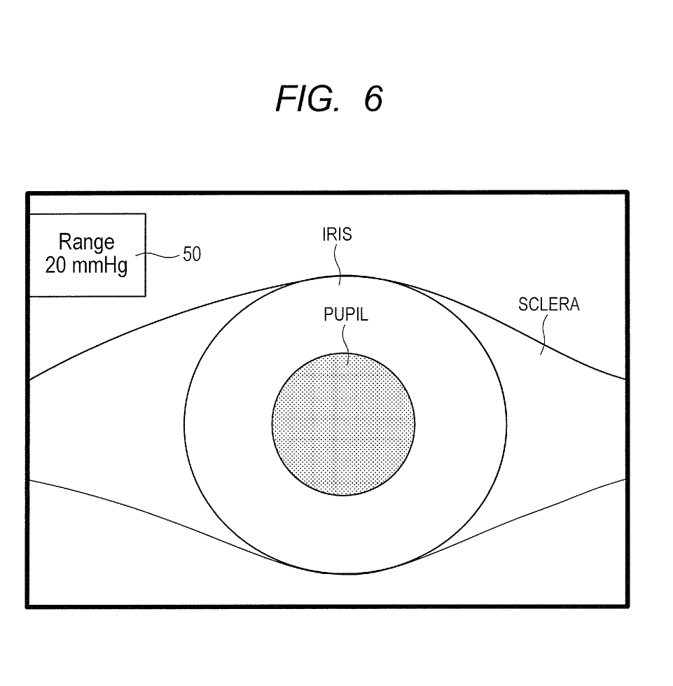

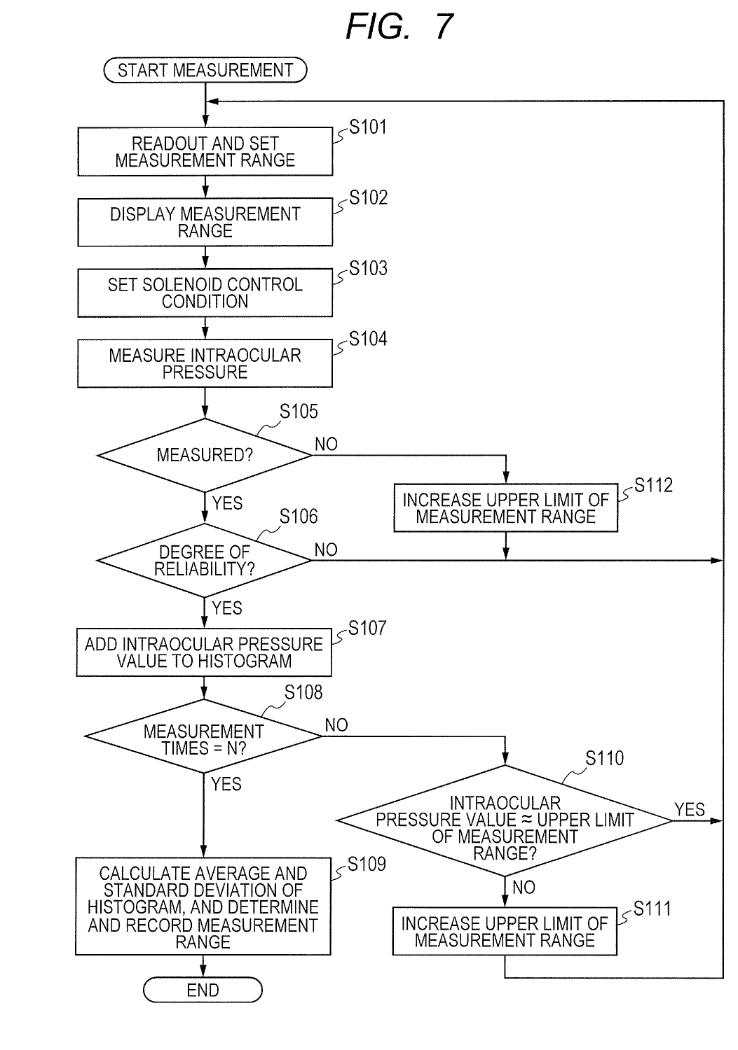

First, determination of the measurement range to be executed through a single intraocular pressure measurement is described with reference to a flow chart of FIG. 5. In Step S01, the system control portion 100 reads out the upper limit value of the measurement range stored in the memory portion 103, and determines the measurement range. In Step S02, the system control portion 100 displays the measurement range on the LCD monitor 106 as illustrated in FIG. 6. The system control portion 100 includes a calculation unit for obtaining statistical data on measurement results of the intraocular pressure via the calculation processing portion 102 and the like, and a condition determination unit for determining, based on the statistical data, the measurement range of the intraocular pressure value stored in the memory portion 103 serving as a storage unit. The system control portion 100 further includes module areas that function as a determination unit for determining the control condition of the fluid ejection unit based on the measurement range determined by the condition determination unit. Note that, the condition determination unit corresponds to the first determination unit, and the determination unit for determining the control condition corresponds to a second determination unit.

In Step S03, the system control portion 100 determines the control condition of the solenoid so as to eject air that allows measurement of an intraocular pressure value corresponding to the upper limit, of the measurement range. In Step S04, the system control portion 100 drives the solenoid 42, and thus compressed air is ejected by the piston 40 toward the cornea Ec of the eye E to be inspected through the nozzle 22. The system control portion 100 stores, in the memory portion 103, a pressure signal detected by the pressure sensor 45 of the air chamber 23 and a received light signal from the light receiving element 34. The system control portion 100 calculates the intraocular pressure value based on the information stored in the memory portion 103 with reference to a peak value of the received, light signal and a pressure signal at the peak. The system control portion 100 displays the calculated intraocular pressure value on the LCD monitor 106.

In Step S05, the system control portion 100 determines whether the measurement is successful or results in an error. When the measurement is successful, in Step S06, the system control portion 100 determines whether the degree of reliability of the measurement value is high or low. The degree of reliability, that is, the appropriateness of reliability is determined by module areas of the system control portion 100 that function as a reliability evaluation unit for determining the appropriateness of reliability through comparison between the measurement value and a preferred range of the measurement value, which is determined based on the measurement range. When the degree of reliability is high, in Step S07, the system control portion 100 adds the measurement value to the histogram recorded in the memory portion 103.

When the system control portion 100 determines that the degree of reliability is low, the statistical data is not stored in the memory portion 103, and the flow returns to Step S01 so that the system control portion 100 reads out the measurement range again, that is, changes and determines the upper limit value thereof. Then, the system control portion 100 executes the measurement of the intraocular pressure value. Note that, when reading out the measurement range again, the system control portion 100 determines a difference between the measured intraocular pressure value and the upper limit value of the measurement range. When the difference is larger than a predetermined value, in the subsequent measurement of the intraocular pressure of the eye to be inspected, the system control portion 100 changes the upper limit value of the measurement range based on the previous measurement result of the intraocular pressure value.

Also when the system control portion 100 determines in Step S05 that the measurement of the intraocular pressure results in an error, the flow returns to Step S01 so that, in the subsequent measurement of the eye to be inspected, the system control portion 100 changes the upper limit value of the measurement range in the case where the measurement results in an error. Then, the system control portion 100 executes the measurement of the intraocular pressure value again.

In Step S08, the system control portion 100 calculates the average and the standard deviation of the histogram, and determines the measurement range having an upper limit value corresponding to the intraocular pressure value obtained by adding the standard deviation to the average. The value of the measurement range is stored in the memory portion 103, and the flow is ended. Further, when the measurement results in an error in Step S05, the system control portion 100 increases the upper limit value of the measurement range by an amount corresponding to the standard deviation, and executes the measurement again through the process from Step S01. Still further, when the system control portion 100 determines in Step S06 that the reliability is low and inappropriate, the system control portion 100 executes the measurement again through the process from Step S01 without adding the measurement result to the histogram.

Next, a procedure of determination of the measurement range to be executed through N measurements is described with reference to a flow chart of FIG. 7. The process of Steps S101 to S107 is executed through the same procedure as in the determination of the measurement range to be executed through a single measurement. In Step S108, the system control portion 100 determines whether or not the number of times of the executed measurement has reached a predetermined number of times of measurement. When the number of times of the executed measurement has reached the predetermined number of times of measurement, in Step S109, the system control portion 100 calculates the average and the standard deviation of the histogram, and determines the measurement range having an upper limit value corresponding to the intraocular pressure value obtained by adding the standard deviation to the average. The value of the measurement range is stored in the memory portion 103, and the flow is ended. When the number of times of the executed measurement has not reached the predetermined number of times of measurement, in Step S110, the system control portion 100 determines whether or not the measured intraocular pressure value is close to the upper limit value of the measurement range. When the measured intraocular pressure value is close to the upper limit value of the measurement range, the system control portion 100 starts the subsequent measurement through the process from Step S101. When the measured intraocular pressure value is not close to the upper limit value of the measurement range, in Step S111, the system control portion 100 decreases the upper limit value of the measurement range based on the measured intraocular pressure value, and executes the subsequent measurement through the process from Step S101. Further, when the measurement results in an error in Step S105, the system, control portion 100 increases the upper limit value of the measurement range by an amount corresponding to the standard deviation, and executes the measurement again through the process from Step S101. Still further, when the system control portion 100 determines in Step S106 that the reliability is low and inappropriate, the system control portion 100 executes the measurement again through the process from Step S101 without adding the measurement result to the histogram. That is, in this example, the system control portion 100 further includes module areas incorporated in the condition determination unit to function as a measurement range designation unit for designating the measurement range. When the system control portion 100 determines as described above that the measurement result is not preferred, the measurement range designation unit maintains the designated measurement range until the number of measurement results of the statistical data that are stored in the memory portion 103 becomes equal to or larger than a predetermined number.

At the time immediately after product shipment, the histogram data is not accumulated in the apparatus, and hence the measurement range is not determined. As a determination method for an initial measurement range, two methods are described below. In the first method, a user designates the measurement range, and executes the measurement within the measurement range until a designated number of pieces of measurement data are accumulated. When the designated number of pieces of measurement data are then accumulated, the process proceeds to the measurement sequence for determining the measurement range based on the histogram as illustrated in FIG. 5 or 7. In the second method, pieces of histogram data obtained through clinical practice at multiple contact lens clinics and hospitals are stored in the apparatus at the time of shipment. The user selects, as initial data, the histogram data close to the condition of the place where the apparatus is installed, to thereby execute the measurement. Further, pieces of measurement data that have been accumulated in the hospital where the apparatus is installed may be input to the apparatus as initial data. That is, in this case, multiple models of the statistical data are stored in the memory portion 103 at the time of shipment of the non-contact tonometer, and the condition determination unit is configured to select one of the multiple models, and to determine the measurement range based on the selected model.

Further, the histogram of the measurement values is stored in this embodiment, but the number of the measurement values, the average of the measurement values, or the standard deviation of the measurement values may be stored instead so as to reduce the capacity of the storage area.

Note that, in the configuration described as an example in this embodiment, the LCD monitor 106 is provided in the same apparatus, but the LCD monitor 106 may be arranged to be separable from the main body. That is, it is preferred that the system control portion 100 include a display control unit for displaying the measurement range, which is determined by the above-mentioned condition determination unit, in a predetermined format on the LCD monitor 106 serving as a display unit in combination with an image of the eye to be inspected, such as an image of the cornea.

Thus, the configuration of the apparatus of this embodiment is detailed through the above description.

In this embodiment, the tonometer is described as an example of the non-contact ophthalmologic apparatus. However, the embodiment of the present invention is not limited thereto, and the present invention may also be realized by a processing apparatus and a processing method for calculating the intraocular pressure value. In this case, the processing apparatus includes the first determination unit and the second determination unit. The first determination unit is configured to determine the measurement range of the intraocular pressure of the eye to be inspected based on the statistical data on the measurement results of the intraocular pressure of the eye to be inspected. Further, the second determination unit is configured to determine, based on the determined measurement range, the control condition of the fluid ejection unit for ejecting compressed air toward the cornea of the eye to be inspected.

(Other Embodiments)

Further, the present invention is also implemented by executing the following processing. Specifically, in this processing, software (program) for implementing the functions of the above-mentioned embodiment is supplied to a system or an apparatus via a network or various kinds of storage medium, and a computer (or CPU, MPU, or the like) of the system or the apparatus reads out and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-047753, filed Mar. 11, 2013, which is hereby incorporated by reference herein in its entirety.

What is claimed is:

1. A non-contact ophthalmologic apparatus, comprising:
a fluid ejection unit for ejecting compressed air toward a cornea of an eye to be inspected;
an intraocular pressure measurement unit for measuring an intraocular pressure of the eye to be inspected by detecting a state in which the cornea is deformed through the ejection of the compressed air;
a storage unit for storing measurement results of the intraocular pressure as statistical data;
a first determination unit for determining a measurement range of the intraocular pressure of the eye to be inspected based on the statistical data; and
a second determination unit for determining a control condition of the fluid, ejection unit based on the determined measurement range.

2. A non-contact ophthalmologic apparatus according to claim 1, wherein, the statistical data is calculated based on a histogram obtained in accordance with the measurement results.

3. A non-contact ophthalmologic apparatus according to claim l, further comprising a display control unit for displaying, on a display unit, the measurement range determined by the first determination unit.

4. A non-contact ophthalmologic apparatus according to claim 1, wherein the storage unit stores a histogram of the measurement results, an average of the measurement results, and a standard deviation of the measurement results as the statistical data.

5. A non-contact ophthalmologic apparatus according to claim 1, wherein the measurement results stored in the storage unit are obtained through control of the control condition determined by the second determination unit.

6. A non-contact ophthalmologic apparatus according to claim 1, further comprising a reliability evaluation unit for determining appropriateness of reliability of the measurement results,
wherein, when the reliability evaluation unit determines that the reliability of the measurement results is low and inappropriate, the storage unit avoids storing the statistical data on the measurement results.

7. A non-contact ophthalmologic apparatus according to claim 1, wherein, when the intraocular pressure measurement unit has failed to measure the intraocular pressure, in subsequent measurement of the eye to be inspected, the first determination unit changes an upper limit value of the measurement range in the case where the intraocular pressure measurement unit has failed to measure the intraocular pressure, and then the intraocular pressure measurement unit measures the intraocular pressure again.

8. A non-contact ophthalmologic apparatus according to claim 1,
wherein the first determination unit determines a difference between a value of the measured intraocular pressure and an upper limit value of the measurement range, and
wherein, when the difference is larger than a predetermined value, in subsequent, measurement of the intraocular pressure of the eye to be inspected, the first determination unit changes the upper limit value of the measurement range based on a previous measurement result of the value of the intraocular pressure.

9. A non-contact ophthalmologic apparatus according to claim 1,
wherein the first determination unit comprises a measurement range designation unit for designating the measurement range, and
wherein the measurement range designation unit maintains the designated measurement range until a number of the measurement results of the statistical data that are stored in the storage unit becomes equal to or larger than a predetermined number.

10. A non-contact ophthalmologic apparatus according to claim 1,
wherein the storage unit stores multiple models of the statistical data at the time of shipment of the non-contact ophthalmologic apparatus, and
wherein the first determination unit selects one of the multiple models, and determines the measurement range based on the selected one of the multiple models.

11. A non-contact ophthalmologic apparatus according to claim 1, wherein the fluid ejection unit comprises:
a piston for compressing air inside a cylinder; and
a solenoid for controlling drive of the piston.

12. A non-contact ophthalmologic apparatus according to claim 11, wherein the control condition of the fluid ejection unit comprises a value of a current allowed to flow through the solenoid.

13. A non-contact ophthalmologic apparatus according to claim 1, further comprising a selection unit for selecting one of multiple types of hospital,
wherein the storage unit stores multiple pieces of statistical data corresponding to the multiple types of hospital, and
wherein the first determination unit determines one of the multiple pieces of statistical data corresponding to the selected one of the multiple types of hospital, and determines the measurement range of the intraocular pressure of the eye to be inspected based on the determined one of the multiple pieces of statistical data.

14. A processing apparatus, comprising:
a first determination unit for determining a measurement range of an intraocular pressure of an eye to be inspected based on statistical data on measurement results of the intraocular pressure of the eye to be inspected; and
a second determination unit for determining, based on the determined measurement range, a control condition of a fluid ejection unit for ejecting compressed air toward a cornea of the eye to be inspected.

15. A control method for a non-contact ophthalmologic apparatus, the non-contact ophthalmologic apparatus comprising:
a fluid ejection unit for ejecting compressed air toward a cornea of an eye to be inspected; and
an intraocular pressure measurement unit for measuring an intraocular pressure of the eye to be inspected by detecting a state in which the cornea is deformed through the ejection of the compressed air,
the control method comprising:
storing measurement results of the intraocular pressure as statistical data;
determining a measurement range of the intraocular pressure of the eye to be inspected based on the statistical data; and
determining a control condition of the fluid ejection unit based on the determined measurement range.

16. A non-transitory tangible medium having recorded thereon a program for causing a computer to perform the steps of the control method according to claim 15.

17. A processing method, comprising:
determining a measurement range of an intraocular pressure of an eye to be inspected based on statistical data on measurement results of the intraocular pressure of the eye to be inspected; and
determining, based on the determined measurement range, a control condition of a fluid ejection unit for ejecting compressed air toward a cornea of the eye to be inspected.

18. A non-transitory tangible medium having recorded thereon, a program for causing a computer to perform the steps of the method according to claim 17.
